(19) United States
(12) Patent Application Publication
(10) Pub. No.: US 2005/0049590 A1
(43) Pub. Date: Mar. 3, 2005

(54) Spinal implant with securement spikes

(76) Inventors: Neville Alleyne, La Jolla, CA (US); James R. Gerchow, Sturgis, MI (US); Philip James Sluder, El Cajon, CA (US); Makoto Nonaka, La Jolla, CA (US); Kendall Bartlett, Chula Vista, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/794,586

(22) Filed: Mar. 5, 2004

Related U.S. Application Data
(60) Provisional application No. 60/453,242, filed on Mar. 7, 2003.

(51) Int. Cl. A61B 17/56

(52) U.S. Cl. 606/61

(57) ABSTRACT

Spinal implants with extending spikes include spikes with laterally extending projections that may form barbs. Several different spike driver mechanisms are also provided. In one embodiment, sliding wedge spike drivers are used. In another embodiment, threaded rotating spike drivers are used. Gear driven spikes may also be provided in some embodiments. Worm gear trains and rack and pinion gear trains may be used to extend the spikes.

### RELATED APPLICATIONS

This application claims priority under 35 U.S.C. § 119(e) from provisional application No. 60/453,242 filed Mar. 7, 2003, which is hereby incorporated by reference in its entirety.

### BACKGROUND OF THE INVENTION

1. Field of the Invention

This invention relates to a spinal implant for placement between two opposing vertebral bodies during a fusion procedure.

2. Description of the Related Technology

A wide variety of interbody devices have been used or proposed for use in vertebral fusion procedures. These are commonly referred to as “cages” and may comprise rings, dowels, fenestrated and/or threaded boxes or cylinders that are placed between the vertebra being fused. Different styles are made from a variety of different materials including titanium, stainless steel, or carbon fiber. Dowels and rings are sometimes constructed of allograft bone. In most cases, the cage is installed into a distracted disk space following disk removal, and cancellous bone chips are implanted in and around the cage between the vertebral bodies. If the procedure is successful, bone tissue permeates the disk space through and around the cage, and a solid bony fusion is formed which rigidly couples the two vertebra.

In spite of a fair number of successes, surgical experience with these cages has at times been fraught with some disasters with the cages migrating within the disk space or with end plate erosions and collapse of the height of the disk space. Studies have also shown widely varying successful fusion rates with this technique.

These problems have led to the augmentation of these devices with pedicle screw fixation with or without posterolateral bone grafting. Although this improves fusion rates, pedicle screw fixation in conjunction with an interbody device can lead to further soft tissue dissection and increased intraoperative bleeding and increase in hospital stay. Furthermore, there is an increase in the risk for infection and for potential nerve injury with pedicle screws breaking through the pedicle and injuring the nerve roots or thecal sac. Furthermore, although the improved clinical outcomes associated with pedicle screw augmentation are significant, combining pedicle screw implantation with cage fusion procedures has also been found to significantly increase the average total cost of the surgery over stand-alone cage fusions.

For these reasons, it would be beneficial for a cage design to be developed which has a high fusion rate without pedicle screw stabilization. However, and although a large number of interbody fusion devices have been developed, none have thus far been demonstrated to eliminate the need for additional pedicle screw fixation to achieve a high fusion success rate.

### SUMMARY OF CERTAIN INVENTIVE ASPECTS

In one embodiment, the invention comprises a spinal implant configured for placement between vertebral bodies comprising a housing and one or more spikes coupled to the housing and configured to couple the housing to the vertebral bodies, wherein at least one of the spikes comprises at least one laterally extending projection for engaging the bone. In some embodiments, the laterally extending projection forms a barb.

In another embodiment, the invention comprise a spinal implant, comprising: a housing defining at least first and second pairs of holes, wherein the first pair of holes are formed on first and second surfaces of the housing, respectively, the second surface opposing to the first surface, and wherein the second pair of holes are formed on the first and second surfaces, respectively. The implant also comprises at least first and second pairs of spikes, the first pair of spikes opposing to each other and the second pair of spikes opposing to each other, each spike having a base portion and a top portion, wherein each spike is configured to reside within the housing in a retracted mode and the top portions of each spike are configured to protrude from the housing via the first and second pairs of holes, respectively, in an extended mode, and wherein each top portion is configured to be inserted into a vertebral body. Furthermore, at least one driver is provided comprising at least two wedge structures mounted to an extended shaft and configured such that a first one of the wedge structures contacts the base portions of the first pair of spikes, and a second one of the wedge structures contacts the base portions of the second pair of spikes so as to transfer each spike from the retracted mode into the extended mode. As with other embodiments, at least one of the spikes may comprise at least one projection on its top portion, and the projection may be barbed.

In another embodiment, a spinal implant comprises certain gear train driven spikes. In one embodiment, a gear train comprising at least a worm gear is coupled to the spike and is configured to transfer the spike from the retracted mode into the extended mode. In another embodiment, a gear train comprising at least a pinion gear is coupled to the spike and configured to transfer the spike from the retracted mode into the extended mode.

In another embodiment, a method of fixing an implant between vertebral bodies is provided. The method comprises inserting the implant between the vertebral bodies, extending spikes coupled to a body of the implant into the vertebral bodies, and using the spikes to pull the vertebral bodies toward the implant body.

### BRIEF DESCRIPTION OF THE DRAWINGS

FIG. 1 illustrates a conceptual drawing that shows how an interbody implant device is used.

FIGS. 2A and 2B are perspective and cutaway views of a spike tip.

FIG. 3A through 3D illustrate an interbody implant device having a sliding wedge spike driver.

FIG. 4 is a cutaway view of an implant with a sliding spike driver having a pair of wedges.

FIG. 5 is a cutaway view of an implant with a threaded rotating spike driver having a pair of wedges.

FIGS. 6A through 6F illustrate an implant having a threaded rotating spike driver with a pair of wedges that move in opposite directions to extend the spikes.

FIGS. 7A through 7C illustrate an implant having two separate threaded rotating spike drivers.

FIGS. 8A through 8C illustrate an implant having a rotating camshaft spike driver.

FIGS. 9A through 9C illustrates an implant having a worm gear train spike driver.

FIG. 10 is a cutaway view of an implant having a pair of jack screw spike drivers comprising threaded worm gear nuts.

FIG. 11 is a cutaway view of an implant having a collinear jack screw spike driver comprising a threaded worm gear nut that drives two spikes simultaneously.

FIGS. 12A through 12C illustrate an interbody implant device having a rack and pinion spike driver.

### DETAILED DESCRIPTION OF CERTAIN EMBODIMENTS OF THE INVENTION

The foregoing and other features of the invention will become more fully apparent from the following description and appended claims taken in conjunction with the following drawings, in which like reference numerals indicate identical or functionally similar elements.

1. Overall Implant Configuration

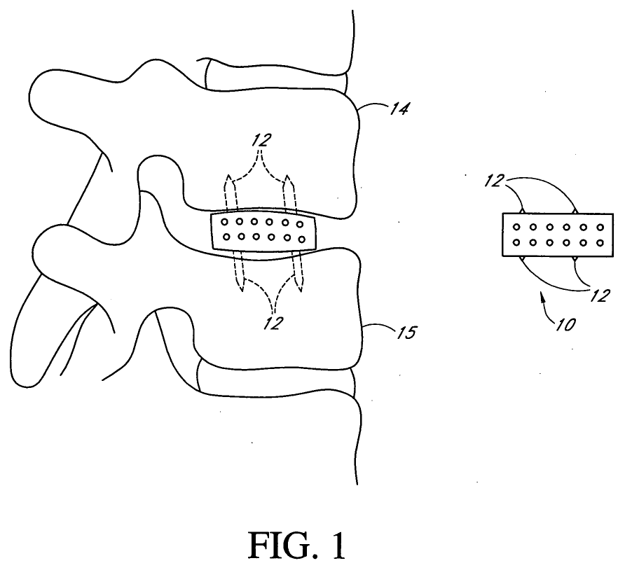

FIG. 1 illustrates an interbody implant device 10 in use. Referring to FIG. 1, the interbody implant device is inserted between vertebral bodies 14, 15 following removal of the spinal disk. FIG. 1 illustrates an anterior approach, but it will be appreciated that posterior insertion methods can be used as well with all invention embodiments described herein, and may in fact be preferable in some cases.

In the embodiment of FIG. 1, the device 10 includes a plurality of spikes 12. The spikes 12 reside within the device 10 in a retracted mode as shown in FIG. 1. The device 10 is inserted into the disk space between two opposing vertebral bodies 14 and 15 as shown in FIG. 1. After the device 10 is inserted, the spikes 12 are extended from the device 10 so as to couple the two vertebral bodies 14 and 15 via the protruded portions of each spike 12.

Throughout the application, the retracted mode represents a state of the interbody device 10 where the spikes 12 are located in the inside of the device 10 as exemplified in the right hand side of FIG. 1. In addition, the extended mode represents a state of the interbody device 10 where the top portions of the spikes 12 are exposed to the outside of the device 10 as exemplified in the left hand side of FIG. 1.

A few devices with expanding spikes such as are illustrated in FIG. 1 have previously been proposed. These include the devices described in U.S. Pat. No. 6,102,950 to Vaccaro and U.S. Pat. No. 5,800,547 to Schafer et al. However, as will be described further below, insufficient attention has been paid to the design of the spikes themselves and also to their method of extension.

Specifically, to help reduce or eliminate the need for supplemental pedical screw fixation, it is advantageous if the vertebral bodies are pulled together to clamp the cage between them as much as possible. No design capable of this function has heretofore been available. In addition, the spike extensions possible with conventional designs is very limited, which in turn limits the amount of stability the spikes can provide.

In some embodiments, the interbody device 10 can be manufactured in a mechanically compressible and expandable manner, in addition to having extendable spikes, allowing it to be placed through a small opening in its compressed state, and once inside the disk space the device 10 can expand in a horizontal direction and also vertically.

It will be appreciated that a wide variety of mechanical methods may be used to produce such an expandable device 10. For example, the expansion mechanism may be a sliding mechanism that can be engaged by using a screw driver to turn and expand the device 10 horizontally. Another set of screws may then be used to turn and increase the vertical height of the device 10 and allow the teeth, anchor, or bolt to clamp and fix the interbody device 10. Additional filling of the device 10 with bone or BMP sponges or DBP sponges, etc., can be done after the device 10 is engaged and expanded to the desired height.

The projections or spikes 12 of the interbody device 10 which extend into the vertebral bodies 14 and 15, can come in many forms. In one embodiment, these projections 12 can come out as fish hooks or anchors which can then penetrate into the end plate to provide stable fixation and compression of the interbody device 10. This will allow an osteo integration with minimal dissection, minimal risk to the nerve root, minimal risk to the thecal sac, minimal bleeding, minimal scar tissue and facilitate shorter hospital stay.

Although not necessary, it can be advantageous to provide a mechanism through which the teeth or other form of anchor is disengageable from the device 10 after installation. For example, expanding teeth that come out of the interbody device 10 can be disengaged from a sleeve that is present in the interbody device 10 so that if there is a need to retrieve the device 10 later, it can be done so through the same minimally invasive technique without having to destroy significantly the vertebral bodies 14 and 15 above and below the interbody device 10.

The interbody device 10 can be made of several materials that are already known in the art of spinal surgery. In one embodiment, the device 100 can utilize material, such as stainless steel, titanium, PGA, PLA, PLLA, tantalum, PMMA, bone, PEEK, or other materials well known and accepted, but not limited to the aforementioned. By having a radiolucent bioabsorbable material the advantages are significant, better ease in radiographic assessment of fusion and osteo integration, and less scatter with MRI. Moreover, there will be a minimal persistent foreign body present in the disk space that should minimize the risk of infection.

In one embodiment, the interbody device 10 provides not only a means for a minimal invasive surgery, expansion within the disk space, both horizontally and vertically, stability within the disk space, easy retrieval of the device 10 in the event of infection or improper placement, but most importantly it allows for platform technology to be utilized for the first time in the disk space without any additional fixation. These end plate projections that penetrate the upper and lower cartilaginous end plates are uniquely designed and encompasses any device or method whereby the interbody device 10 can be anchored and compressed within the disk space. The geometry of the device 10 may be somewhat trapezoidal or rectangular, but is not limited to these geometric shapes. Cylindrically shaped devices could also be anchored in such a fashion. Referring to drawings, different embodiments will be discussed below in more detail.

2. Spike Design

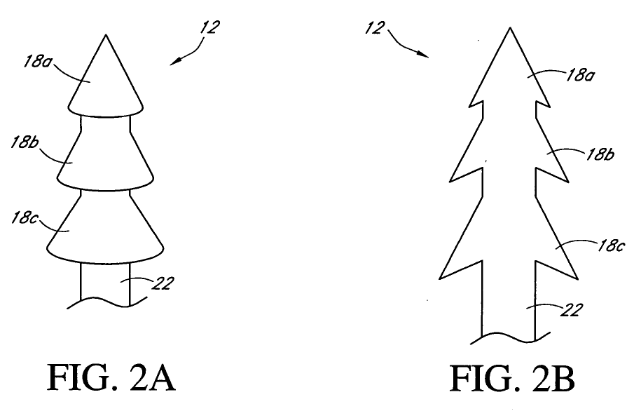

FIG. 2 illustrates a tip design for cage spikes in accordance with one embodiment of the invention. The spikes include at least one projection 18a, 18b, 18c that extends outward from the base diameter of the main spike shaft 22. In some embodiments, such as is shown in FIG. 2, a plurality of conical projections are provided such that the spike tip forms a Christmas tree type configuration. The projections may increase in diameter toward the bottom of the shaft 22, or they may stay the same. It is also advantageous if the bottom surface of the projection slopes upward from the bottom edge or tip of the projection. In this way, the projections can form one or more downwardly projecting barbs. These can engage the vertebral bone like a fish-hook, for example.

If this type of spike design is utilized, the bone tissue will tend to enclose and surround the projections, pulling the vertebral bodies toward the implant, and resisting relative motion between the implant and the vertebral bodies. Furthermore, the normal load on the spine will tend to implant the spikes deeper into the bone, which will not be completely relaxed upon reduction of loading because of the projections. Thus, the spike design leads to progressive enhancement of the attachment between the device 10 and the vertebral bodies 14, 15. This significantly improves fixation, inhibits device migration, and enhances the success of the fusion. As will be further described below, in some embodiments it is advantageous to spring bias the spikes toward the center of the implant. This can further produce a pulling/clamping effect between the vertebra and the implant that enhances stability.

The remaining Figures illustrate conventional conical tapered tip spikes, but it will be appreciated that spikes in accordance with the above description may advantageously be utilized with all of the different cage embodiments described below.

3. Sliding Wedge Drivers

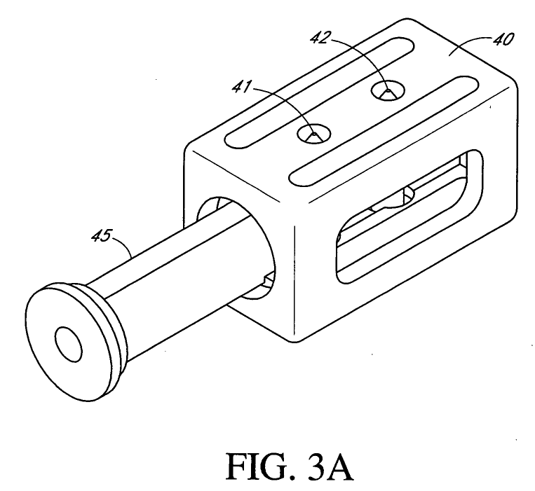

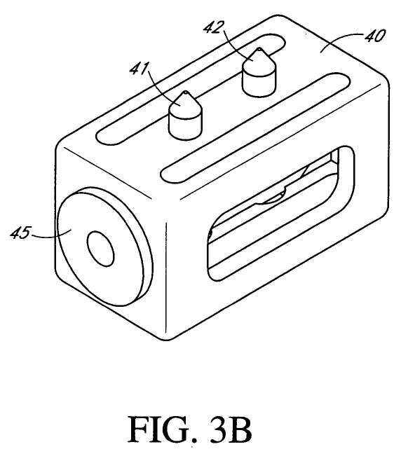

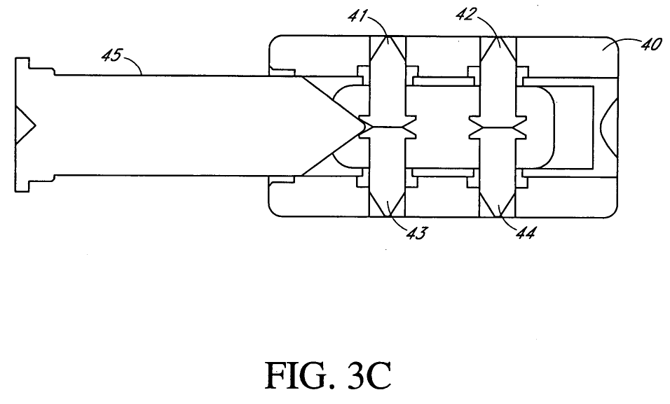

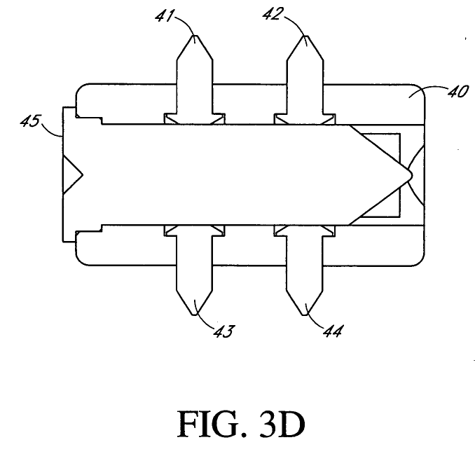

FIGS. 3A through 3D illustrate an interbody implant device having a sliding wedge driver according to one embodiment of the invention. This device comprises a housing 40, a driver 45, and spikes 41-44. FIGS. 3B and 3D illustrate an extended mode where the spikes 41-44 are extended from the housing 40. FIGS. 3A and 3C illustrate a retracted mode where the spikes 41-44 reside within the housing 40.

The spikes 41-44 are captured in holes in the housing 40. The driver 45 has a tapered tip which engages the heads of the spikes. To install the spikes, the driver 45 is forced into the housing 40, and the tapered surface of the driver tip forces the spikes outward. The driver is preferably tapped into the housing with a hammer or mallet without the need for any rotating or threaded engagement with the housing. During surgery, it has been found that such non-rotating, non-threaded methods of engagement are often easier to perform than thread based, rotating engagement designs, and this is one advantage of the system llustrated in FIG. 3.

Thus, referring to FIGS. 3A and 3C, in the retracted mode, most part of the driver 45 is located outside of the housing 40 such that the driver 45 does not push against the base portions of the spikes 41-44. On the other hand, referring to FIGS. 3B and 3D, in the extended mode, the spike 45 slides into the inside of the housing 40 such that the screw 45 pushes against the base portions of the spikes 41-44 until the spikes are exposed to the outside of the housing 40.

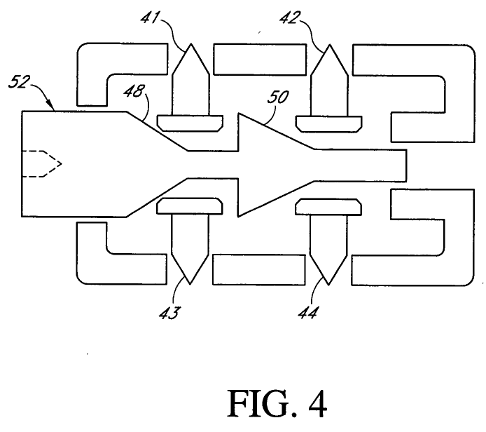

FIG. 4 shows another embodiment of a non-rotating driven wedge design with two wedge portions 48, 50 on the driver 52. In this embodiment, the front wedge 48 is used to engage the front spikes 41, 43 and the rear wedge 50 is used to engage the rear spikes 42, 44. Wedge position on the driver 52 can be configured such that all four spikes are forced outward at the same time, or, for example, the front two spikes could be driven in before the rear wedge 50 contacts and engages the rear spikes.

4. Threaded Rotating Wedge Drivers

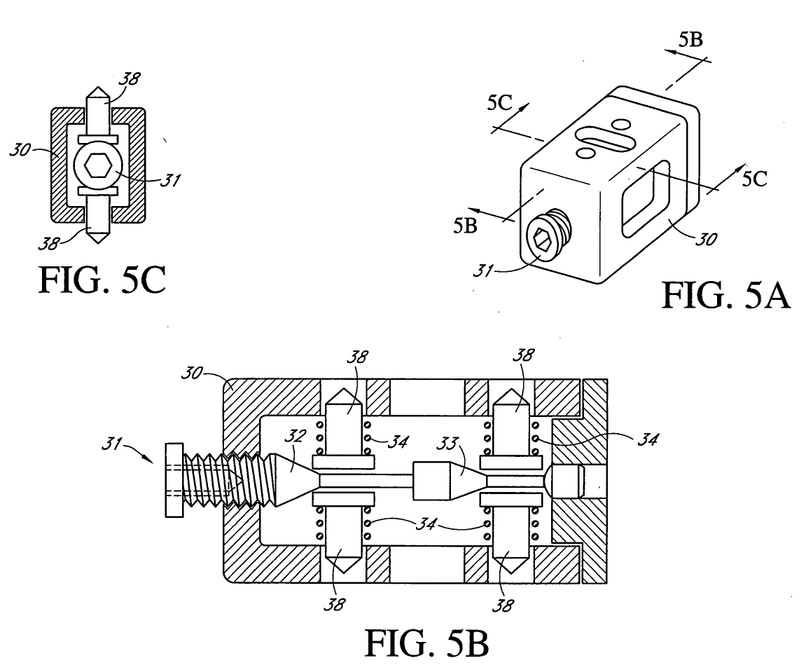

Although there are some disadvantages to a threaded rotating coupling between the housing and the driver, it will be appreciated that the embodiments of FIGS. 3 and 4 could utilize such a design. Another example of a threaded spike driver is illustrated in FIG. 5.

FIG. 5 illustrates an interbody implant device having two wedge structures, moving in the same direction, according to another embodiment of the invention. The device of FIG. 3 comprises a housing 30, a driver 31, two wedges 32 and 33, and four spikes 38. A cap 36 is placed on one side of the housing 30 to close the side of the housing 30 in this embodiment. FIG. 5 only shows a retracted mode of the device. The extending operation of the spikes 38 by the wedges 32 and 33 is substantially the same as that of FIGS. 3 and 4, except the driver 31 is externally threaded, and rotates within an internally threaded channel 37 in the housing 30.

The driver 31 is inserted into the housing 30 such that the rotation of the driver 31 forces the wedges to move to the right by rotating the driver 31.

In order to transfer the spikes 38 from the retracted mode to the extended mode, the driver 31 is turned, for example, clockwise such that each wedge 32 and 33 rotates, moves to the right in FIG. 5 and pushes against the base portions of each spike 38 until the top portions of each spike 38 protrude from the housing 30. Again, two opposing vertebral bodies are coupled to each other via the protruded top portions of each spike 38.

It is one feature of the embodiments of FIGS. 3, 4, and 5 that the driver 45, 52, or 31 is removable after the spikes are set into the vertebral bodies. This leaves room inside the cage housing for the introduction of cancellous bone for fusion.

The embodiment of FIG. 5 also illustrates the use of compression springs 34 which can be provided to produce a pulling force on the vertebral bodies toward the cage housing. These may be provided in any of the cage embodiments described herein, and are especially useful when used in conjunction with the barbed spike design illustrated in FIG. 2. If the springs are strong enough, they can be used to retract the spikes if the device needs removal. In these embodiments, either the driver remains in the implant, or another latch which maintains the spikes in an extended position is provided.

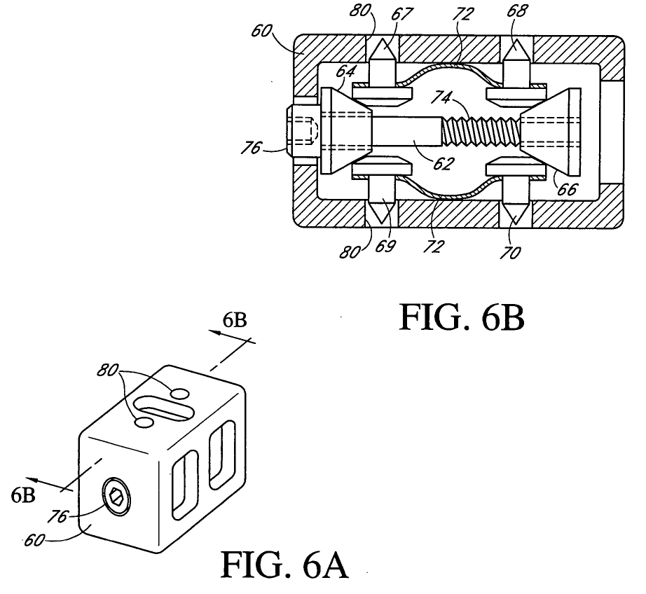

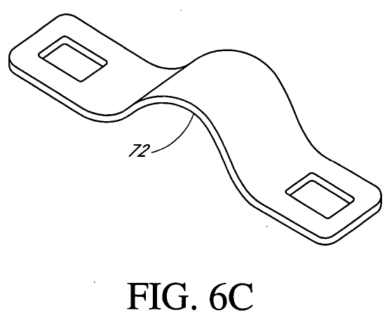

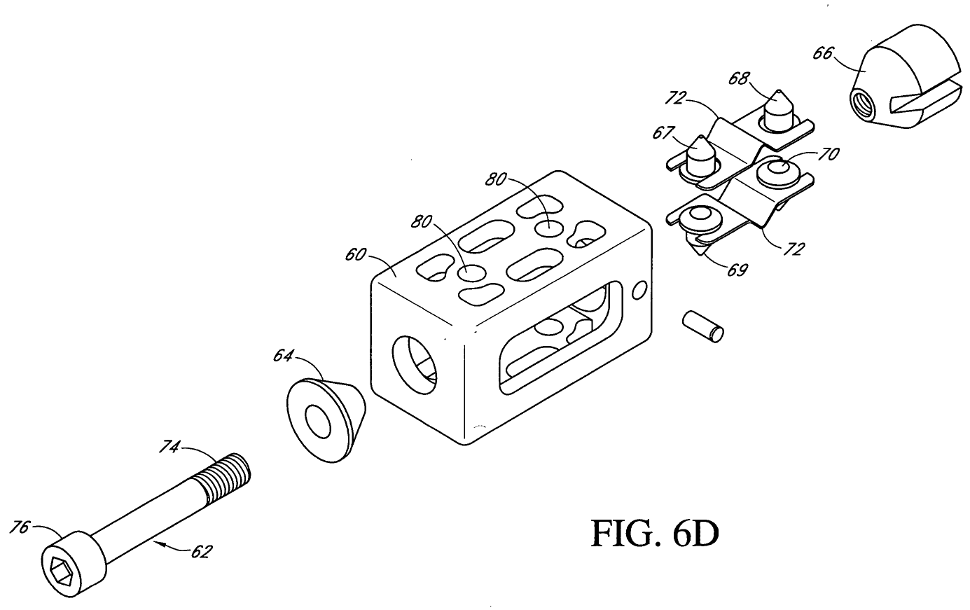

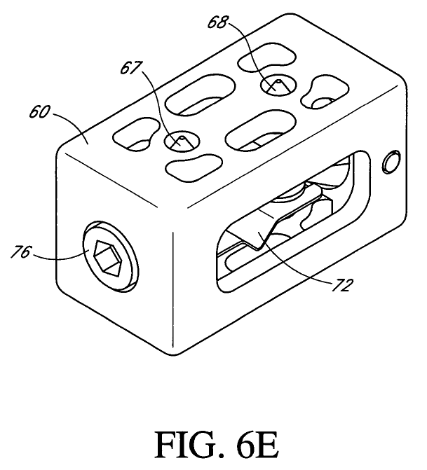

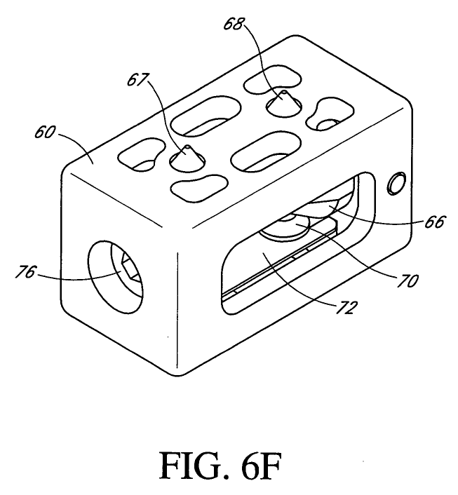

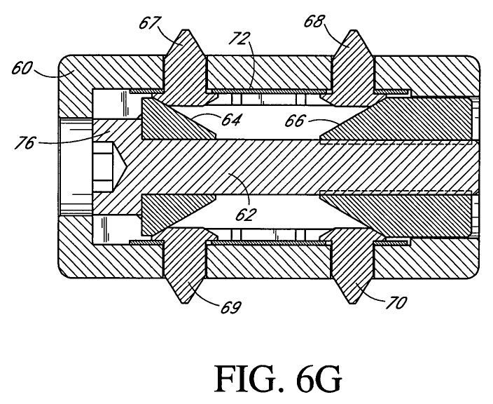

FIG. 6A through 6F illustrate an interbody implant device having two wedge structures, moving in opposite directions, according to one embodiment of the invention. The device of FIG. 6 comprises a housing 60, a rotating shaft 62, wedges 64 and 66, and spikes 67-70. FIG. 6A illustrates a cross sectional view of the implant device. FIGS. 6E and 6F illustrate an extended mode where the spikes 67-70 are extended from the housing 60. FIGS. 6A and 6D illustrate a retracted mode where the spikes 67-70 reside within the housing 60.

Referring to FIGS. 6A-D, the retracted mode will be explained. As shown in these Figures, in the retracted mode, the spikes are located inside the housing. Each spike comprises a base portion and a top portion. In this embodiment, the spikes 67-70 are biased inward toward the center of the housing by a spring 72 that has a flexing central convexly bowed region that is pressed against the inside of the housing. The ends of the spring are coupled to a respective spike base such that the central bowed portion tends to pull the spikes inward. A perspective view of a suitable spring is shown in FIG. 6B.

The shaft 62 is coupled to the wedges 64 and 66. In one embodiment, the shaft 62 comprises a threaded portion 74 and a head portion 76. The front wedge 64 has a throughhole and the rear wedge 66 has an internally threaded portion therein. The shaft is inserted into the throughhole of the wedge 64 until the wedge 64 reaches the head portion 76 as shown in FIG. 6A. The threaded portion 74 of the shaft is adapted to be inserted into and engaged with the threaded portion of the rear wedge 66.

Referring to FIGS. 6E and 6F, the extended mode will be explained. As shown in these Figures, in the extended mode, the top portions of the spikes 67-70 protrude from the housing 60 via openings 80. In order for the spikes to move from the retracted mode to the extended mode, the shaft is rotated, for example, clockwise, such that each wedge 64 and 66 pushes against the base portions of each spike until the top portions of each spike protrude from the housing 60 via the openings 80 respectively.

Specifically, the threaded portion 74 of the shaft rotates clockwise in the inside of the wedge 66. Since the shaft motion to the right in FIG. 6A is limited by the front wedge 64, the wedge 66 moves in the left direction along the threaded portion 74 of the shaft. The wedge 64 also moves in the right direction, clamping the spike bases between the tapered surfaces of the wedges.

Thus, each of the wedges 64,66 moves in the right and left directions, respectively, and pushes against the base portions of each spike until the top portions of each spike are exposed to the outside of the housing 60 via the holes 80.

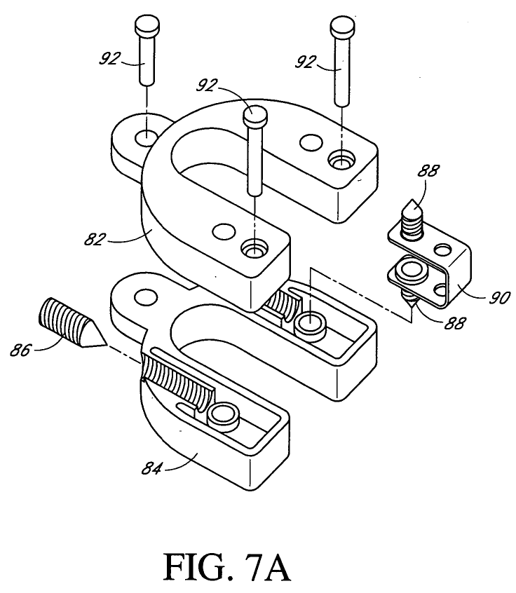

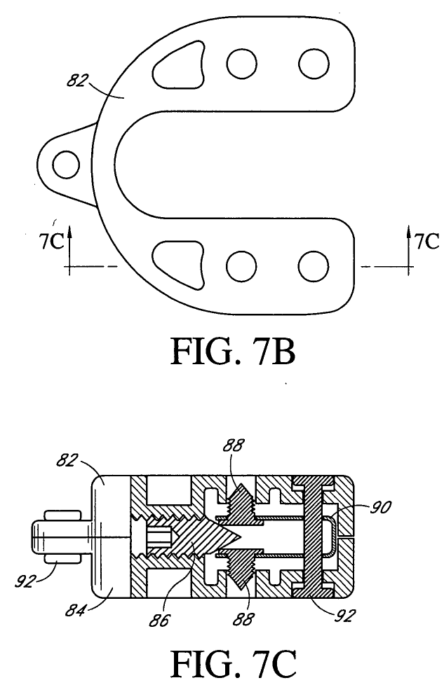

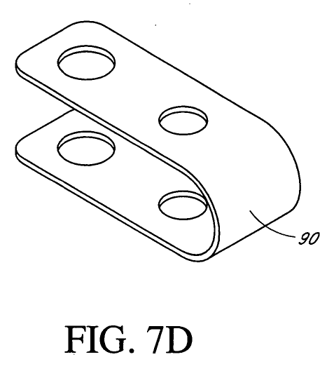

FIG. 7A through 7C illustrate an interbody implant device having a threaded screw structure according to another embodiment of the invention. The device of FIG. 7 comprises upper and lower housing halves 82, 84, a pair of threaded wedge drivers 86 and two pairs of spikes 88. Each pair of spikes 88 are coupled to a hair pin spring 90 (see also FIG. 7C) as shown in FIG. 7A. The upper housing half 82 and lower housing half 84 are connected to each other via rivets 92 to form a complete housing. This overall design may be advantageous in some cases because the housing forms a horseshoe shape with an open central region that provides space for placement of cancellous bone chips for vertebral fusing.

FIG. 7A illustrates an exploded view of the implant device. FIG. 7B illustrates a cross sectional view of the device in a retracted mode where the spikes 88 reside within the housing. To extend the spikes, each driver 86 is turned, for example, clockwise and moves inward via the threaded portions of the drivers 86 into threaded openings in the housing such that each of the drivers 86 pushes against the base portions of each spike 88 until the top portions of each spike protrude from the case. It will be appreciated that a sliding driver design such as the one illustrated in FIG. 3 could also be used.

5. Cam Shaft Drivers

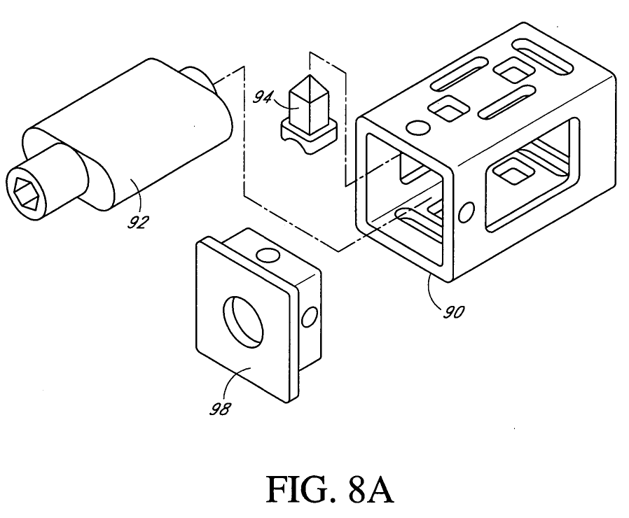

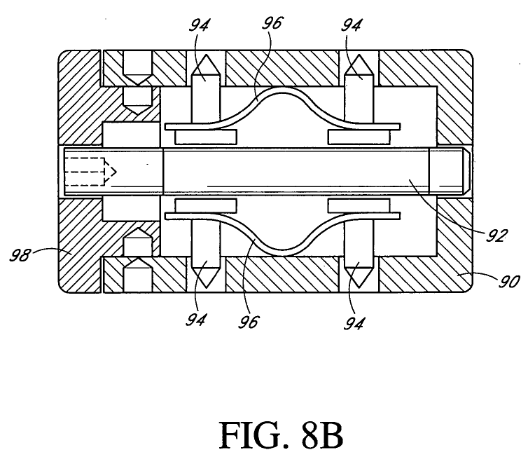

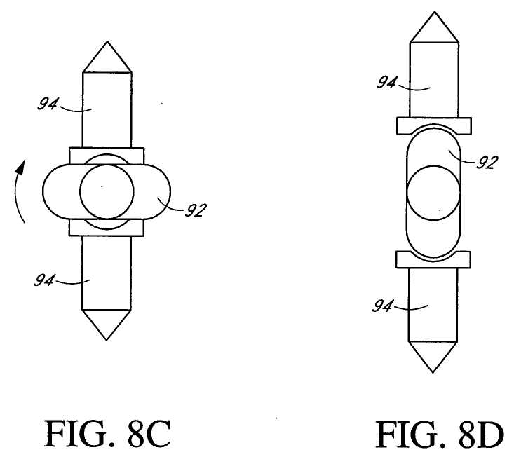

FIGS. 8A through 8C illustrate an interbody implant device having a cam shaft structure according to another embodiment of the invention. The device of FIG. 8 comprises a housing 90, a cam shaft 92 and spikes 94. FIG. 8A illustrates an exploded view of the implant device. FIG. 8B illustrates a retracted mode where the spikes 94 reside within the housing 90.

In one embodiment, each pair of spikes is coupled to springs 96 similar to the embodiment of FIG. 6. After the cam shaft 92, the spikes 94 and the springs 96 are positioned in the housing 90, a cap 98 is placed on one side of the housing to close the housing.

In one embodiment, the cam shaft 610 has two orientations corresponding to whether the spikes 94 are in the extended mode or in the retracted mode as shown in FIG. 8C. The cam shaft 92 is located between the base portions of the spikes in the both of the extended and retracted modes. In the embodiment of FIG. 8, turning the cam shaft 92 about 90° either clockwise or counterclockwise can transfer the spikes 94 from the retracted mode to the extended mode, and vice versa.

6. Worm Gear Train Drivers

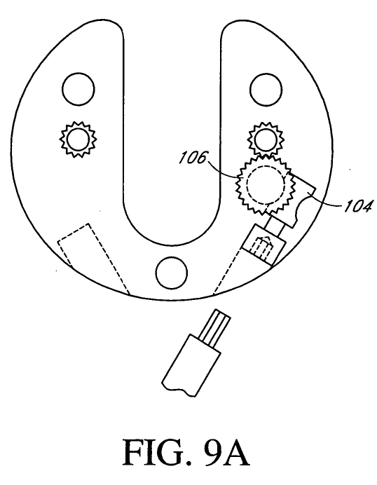

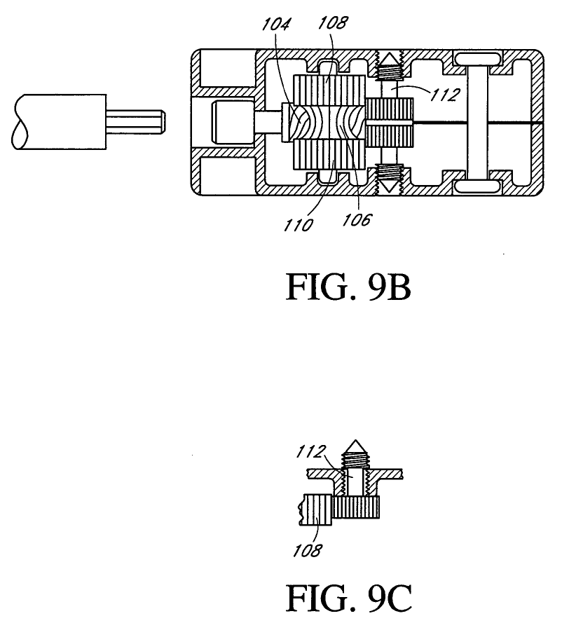

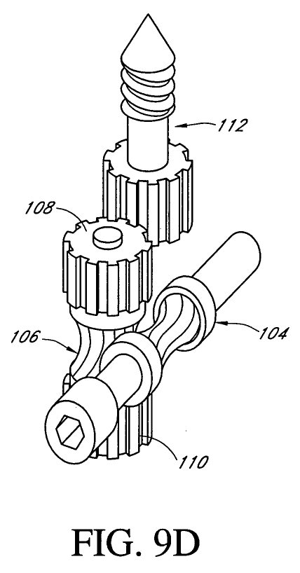

FIGS. 9A through 9C illustrates an interbody implant device having a gear assembly structure according to one embodiment of the invention. The device of FIG. 9 comprises a housing 102 similar to that described above with reference to FIG. 7. However, instead of a moving wedge type driver for the spikes, a gear train is utilized. In this embodiment, a worm gear system is used.

Referring again to FIGS. 9A through 9C, a worm gear 104 rotates along a horizontal axis and meshes with a second worm gear 106 that rotates along a vertical axis. Spur gears 108, 110 are provided, attached above and below the vertical rotating worm gear 106. In one embodiment, a hex driver may be used to turn the horizontal worm gear 104.

This gear assembly which is engaged with the spikes 112 that are in this embodiment provided with geared heads to engage the spur gears 108, 110 such that the spikes rotate in response to worm gear train rotation. The top portion of the spikes are externally threaded and engage internally threaded openings in the housing. Spike rotation then causes the spikes to move outward into the extended position.

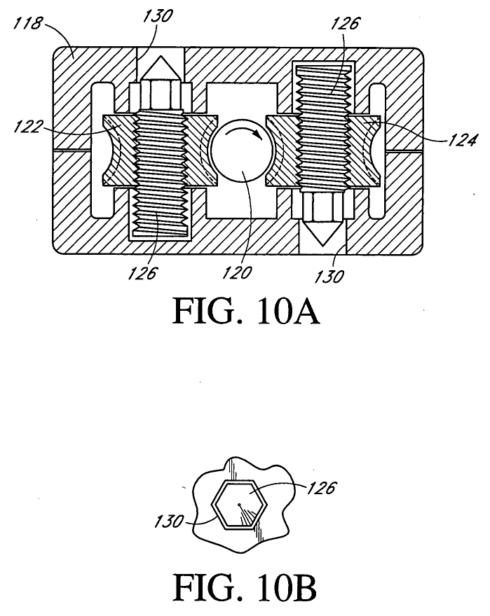

FIG. 10 illustrates a second worm gear train embodiment which uses a jackscrew drive to extend the spikes. The device of FIG. 10 comprises a housing 118, a worm gear 120 rotating on a horizontal axis, and internally threaded worm gear nuts 122, 124 rotating on vertical axes. This embodiment also includes externally threaded spikes 126 positioned inside the worm gear nuts 122, 124. The worm gear 120 is engaged with both of the worm gear nuts 122 and 124 such that the rotation of the worm gear 120 forces the worm gears 122 and 124 to rotate at the same time but in opposite directions.

In these embodiments, the spikes are prevented from rotating by being made in a hex shape and residing in mating hex shaped openings 130 in the housing 118. Because the spikes are held rotationally fixed as the worm gear nuts turn, the threaded coupling between the worm gear nuts and the spikes pushes the spikes out of the housing as the worm gear nuts rotate. The worm gear nut 122 is configured such that its rotation moves the spike in an upper direction as shown in FIG. 10. The worm gear nut 124 is configured such that its rotation moves the spike in a lower direction as shown in FIG. 10.

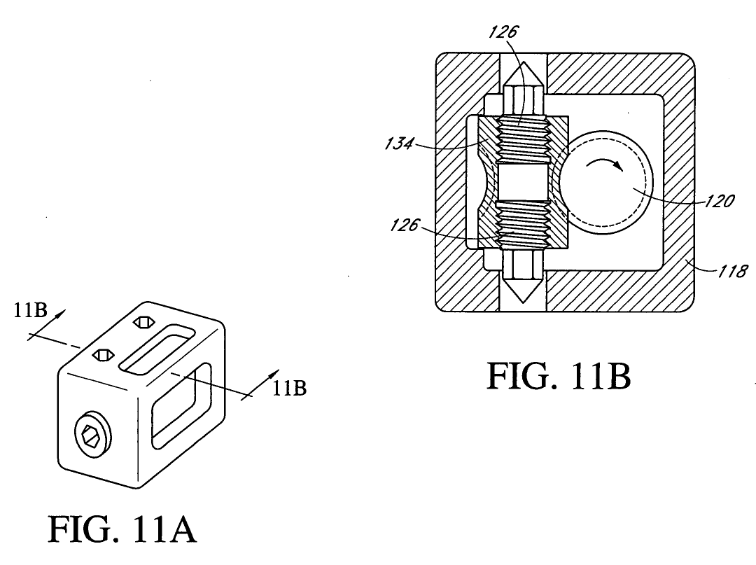

FIG. 11 is a similar embodiment as FIG. 10, except the jackscrew is co-linear, with two opposed spikes residing inside one worm gear nut 134. In this embodiment, the threads on opposite spikes are configured oppositely, such as right hand threads on the bottom spike and left hand threads on top spike so that the single worm gear nut rotation forces the spikes to move in opposite directions.

7. Rack and Pinion Gear Train Drivers

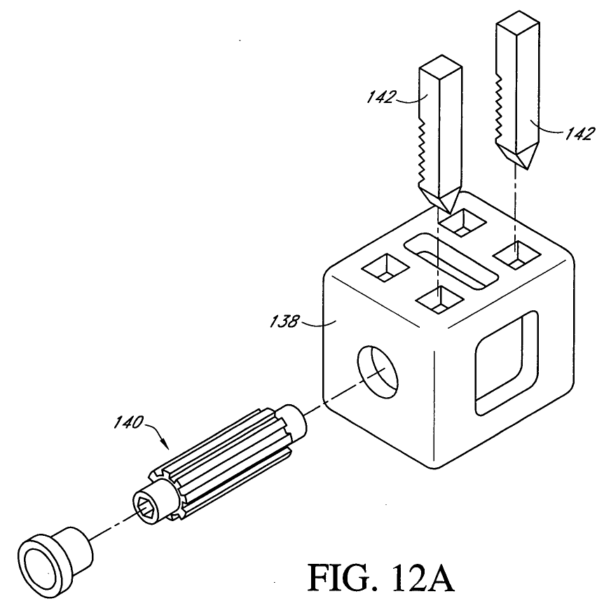

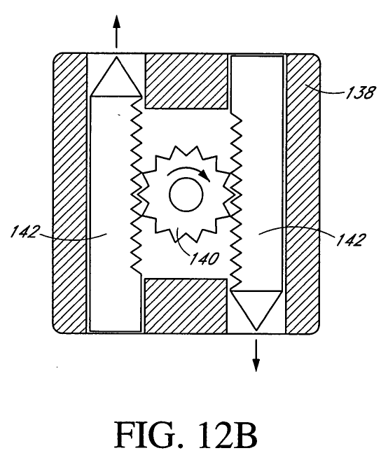

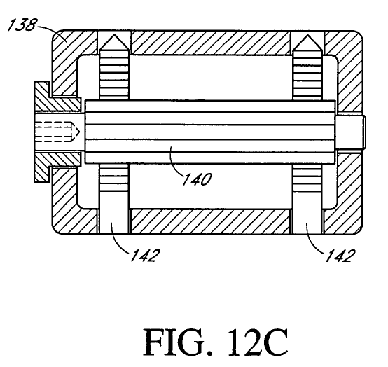

FIG. 12A through 12C illustrate an interbody implant device having a rack and pinion structure according to another embodiment of the invention. The device of FIG. 12 comprises a housing 138, a pinion gear 140 and spikes with racks 142. Each of the spikes 142 has a rack which is configured to be engaged with the pinion gear 140.

Referring to FIG. 12B, the pinion gear 140 is engaged with the rack spikes 142 and by its rotation configured to move the spikes linearly, for example, upward or downward as shown in FIG. 12B. In one embodiment, the pinion gear 140 is coupled to two pairs of rack spikes 142 as shown in FIGS. 11A and 11B. Each pair of spikes moves in opposite directions, for example, upward and downward, respectively, by the rotation of the pinion gear 140. In another embodiment, the pinion gear 140 is coupled to more than two pairs of rack spikes and configured to move all of the rack spikes linearly.

While the above description has pointed out novel features of the invention as applied to various embodiments, the skilled person will understand that various omissions, substitutions, and changes in the form and details of the device or process illustrated may be made without departing from the scope of the invention. Therefore, the scope of the invention is defined by the appended claims rather than by the foregoing description. All variations coming within the meaning and range of equivalency of the claims are embraced within their scope.

1. A spinal implant, comprising:
a housing defining at least first and second pairs of holes, wherein the first pair of holes are formed on first and second surfaces of the housing, respectively, the second surface opposing to the first surface, and wherein the second pair of holes are formed on the first and second surfaces, respectively;
at least first and second pairs of spikes, the first pair of spikes opposing to each other and the second pair of spikes opposing to each other, each spike having a base portion and a top portion, wherein each spike is configured to reside within the housing in a retracted mode and the top portions of each spike are configured to protrude from the housing via the first and second pairs of holes, respectively, in an extended mode, and wherein each top portion is configured to be inserted into a vertebral body;
at least one driver comprising at least two wedge structures mounted to an extended shaft and configured such that a first one of said wedge structures contacts the base portions of said first pair of spikes, and a second one of said wedge structures contacts the base portions of said second pair of spikes so as to transfer each spike from the retracted mode into the extended mode.

2. The implant of claim 1, wherein said driver slidably engages said housing.

3. The implant of claim 1, wherein said driver threadably engages said housing.

4. The implant of claim 1, wherein said wedge structures are integrally formed with said extended shaft.

5. The implant of claim 1, wherein at least one of said wedge structures is slidably attached to said shaft.

6. The implant structure of claim 1, wherein one of the wedge structures has a throughhole and the other wedge structure has a first threaded portion, and the extended shaft has a second threaded portion, and wherein the shaft is configured to be inserted into the one wedge structure via the throughhole and coupled to the other wedge structure via the threaded portions.

7. The implant structure of claim 1, wherein the at least two wedge structures are configured to move in opposite directions to extend said spikes.

8. The implant structure of claim 1, wherein one of the two wedge structures is located between the first pair of spikes and the second pair of spikes in the retracted mode.

9. The implant structure of claim 1, additionally comprising one or more springs configured to bias said spikes toward the center of said housing.

10. The implant of claim 1, wherein at least one of said spikes comprises at least one projection on its top portion.

11. The implant of claim 10, wherein said projection is barbed.

12. A spinal implant comprising:
a housing defining at least one opening;
at least one spike positioned in said opening and configured to be retracted into said housing or extended from said housing through said opening;
a gear train comprising at least a worm gear coupled to said spike and configured to transfer said spike from the retracted mode into the extended mode.

13. The implant of claim 12, wherein the base portion of said spike comprises a spur gear.

14. The implant structure of claim 12, wherein the top portions of each spike progressively extend by rotation of the worm gear.

15. The implant of claim 12, wherein the base portion of said spike is externally threaded.

16. The implant structure of claim 12, comprising a pair of opposed spikes and a pair of opposed openings.

17. A spinal implant comprising:
a housing defining at least one opening;
at least one spike positioned in said opening and configured to be retracted into said housing or extended from said housing through said opening;
a gear train comprising at least a pinion gear coupled to said spike and configured to transfer said spike from the retracted mode into the extended mode.

18. The implant of claim 17, wherein said spike comprises a rack and said rack is coupled to said pinion gear.

19. A spinal implant configured for placement between vertebral bodies, said implant comprising:
a housing; and
one or more spikes coupled to said housing and configured to couple said housing to said vertebral bodies, wherein at least one of said spikes comprises at least one laterally extending projection for engaging said bone.

20. The implant of claim 19, wherein said laterally extending projection forms a barb.

21. The implant of claim 19, comprising a plurality of laterally extending projections.

22. The implant of claim 21, wherein said projections increase in lateral extension from the tip of said spike toward the base of said spike.

23. A method of fixing an implant between vertebral bodies comprising:
inserting said implant between said vertebral bodies;
extending spikes coupled to a body of said implant into said vertebral bodies;
using said spikes to pull said vertebral bodies toward said implant body.

24. The method of claim 23, wherein at least one of said spikes comprises at least one laterally extending projection.

25. The method of claim 24, wherein said spikes are spring biased toward the center of said implant body.
